(19) United States
(12) Patent Application Publication
(10) Pub. No.: US 2018/0346962 A1
(43) Pub. Date: Dec. 6, 2018

(54) NANOSTRUCTURES WITH CATALYTIC ACTIVITY

(71) Applicant: AIT AUSTRIAN INSTITUTE OF TECHNOLOGY GMBH, Vienna (AT)

(72) Inventor: Ivan Barisic, Vienna (AT)

(21) Appl. No.: 15/779,042

(22) PCT Filed: Nov. 25, 2016
(86) PCT No.: PCT/EP2016/078854

§ 371 Date: May 24, 2018

(30) Foreign Application Priority Data
Nov. 27, 2015 (EP) 15196820.3

(51) Int. Cl. C12Q 1/68; C07H 21/00

(52) U.S. Cl. CPC C12Q 1/68; B82Y 5/00; C07H 21/00

(57) ABSTRACT

Provided herein is a nanostructure comprising a nucleic acid scaffold and at least one functional core. The nanostructure is of any two-dimensional or three-dimensional shape such as a sheet, square, rectangle, nanotube, cylinder, ring, disc, ribbon, box, cube, pyramide and rod formed by a DNA and/or RNA scaffold and may have various catalytic activities.

### FIELD OF THE INVENTION

The present invention relates to nanostructures comprising a nucleic acid scaffold and functional core with a catalytic center and methods of using same.

### BACKGROUND

Nanomaterials with predefined size and geometry and various functionalities provide a versatile tool ranging from delivery agents, nano-circuits, sensors and the like in areas from biomedicine to electronics.

Biomolecules including nucleic acids and proteins have an exceptional capability to self-assemble into complex and sophisticated structures such as enzyme complexes or ribosomes. This capability has led to the development of DNA nanotechnology and to the construction of high-resolution and robust artificial DNA nanostructures with a wide variety of shapes, geometries and functions. DNA assembly strategies were initially based on the association of many short oligonucleotides to create a larger structure. Further methods were based on the controlled folding of a long single DNA strand. This “DNA origami” technique has significantly spurred the design of two and three-dimensional DNA structures and has been further refined. Specifically, a DNA origami method using numerous short single strands of DNA to direct the folding of a long single strand of DNA into a desired shape was developed by Rothemund (US20070117109) and led to the generation of nanoscale devices, systems and enzyme factories.

Construction of 1D origami nanoribbons and nanotubes was also used as template for the attachment of a pair of enzymes, resulting in a high-efficiency nanoscale bioreactor (Fu et al., J. Am.Chem. Soc. 135:696-702 (2013)). Further, self-assembled DNA structures were described to serve as scaffolds for secondary chromophore molecules with light-harvesting properties (Pan et al., Nucleic Acids Research 42(4):2159-2170 (2014)) and a programmable antenna array on a DNA origami platform as light harvesting network for use in novel solar cell technologies was presented by Hemmig et al. (Nano Lettters 16 (4):2369-2374 (2016)). Biohybrid photoelectrochemical devices using a light harvesting complex for trapping and converting incident light to electrochemical energy were disclosed in US2014/0042407.

DNA origami was further employed for the formation of complexes for sequestering and binding or processing substances or pathogens using an active moiety surrounded by a nucleic acid scaffold as disclosed in WO2013030831.

However, there is still a great need for the development of protein mimicries in the creation of artificial enzymes with respect to several disciplines, such as biotechnology, biomedical manufacturing and/or in the energy sector.

### SUMMARY

Provided herein is a nanostructure comprising a nucleic acid scaffold and at least one functional core. In one aspect, provided herein is a nanostructure comprising a nucleic acid scaffold and at least one functional core forming a catalytic center, wherein the functional core comprises a nucleic acid molecule with at least one chemically modified nucleotide, preferably a chemically modified nucleotide with one or more amino acid or amino acid analog residue(s). In some embodiments, the nucleic acid scaffold of the nanostructure described herein is a two-dimensional or three-dimensional shape selected from the group consisting of a sheet, square, rectangle, nanotube, cylinder, ring, disc, ribbon, box, cube, pyramide, cross and rod.

In some embodiments, the scaffold is a DNA scaffold. Specifically, the DNA scaffold is assembled by a single-stranded DNA backbone chain and/or at least 50 single-stranded DNA staple chains. In some embodiments, the scaffold is a RNA scaffold. Specifically, the RNA scaffold is assembled by a single-stranded RNA backbone chain and/or at least 50 single-stranded RNA staple chains. In some embodiments, the backbone chain (e.g. RNA or DNA backbone chain) comprises at least 1000 nucleotides. In some embodiments, the staple chains (e.g. DNA or RNA staple chains) comprise at least 30 nucleotides.

In some embodiments, the functional core comprises a catalytic center of an ATP-driven motor, preferably the archaeal rotary motor, or a catalytic center of an enzyme, preferably an ion pump, a light-harvesting complex or a photosystem. In some embodiments, the functional core comprises a catalytic center with at least 5 amino acid residues and/or amino acid analogs. Specifically, the functional core is embedded in the nucleic acid scaffold, preferably the nucleic acid scaffold is a nanotube. In some embodiments, the functional core is bound to the nucleic acid scaffold via staple chains.

Further provided herein is a nanostructure comprising at least one functional core which is embedded in an interior space of a nucleic acid scaffold having the shape of a nanotube, cylinder, pyramide or box. In some embodiments, the at least one functional core comprises a catalytic center with at least 5 amino acid residues and/or amino acid analogs. In some embodiments the nanostructure comprises at least three functional cores (any one of 3, 4, 5, or 6) forming the catalytic center of an ATPase, preferably the ATPase FlaI (e.g. FlaI of Sulfolobus acidocaldarius) embedded in an interior space of a nucleic acid scaffold having the shape of a nanotube, cylinder, pyramide or box. In some embodiments, the nucleic acid scaffold emulates one or more structural or functional proteins of a flagellum or archaellum or fragments thereof, preferably it emulates one or more proteins of FlaI, FlaX, FlaH and FlaJ or fragments thereof (e.g. Sulfolobus acidocaldarius). In some embodiments, the nanostructure further comprises one or more structural or functional proteins of a flagellum or archaellum, preferably it further comprises any one of the proteins FlaI, FlaX, FlaH and/or FlaJ, or fragments thereof (e.g of Sulfolobus acidocaldarius).

Further provided herein is a molecular motor, a valve, or a solar panel comprising the nanostructure as described herein.

### BRIEF DESCRIPTION OF THE DRAWINGS

FIG. 1. Schematic concept of a nanostructure as described herein, an enlargement of its functional core and catalytical center. The components are almost completely made out of DNA and comprise only chemical modifications such as e.g. amino acids and amino acid analogs at critical positions necessary to transform the chemical energy into motion and/or the hydrolysis of a substrate.

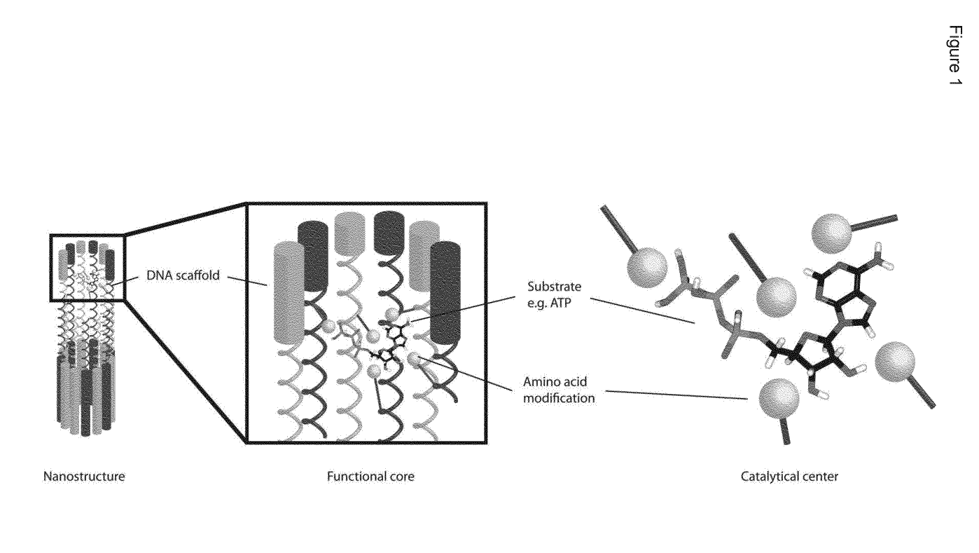

FIG. 2. Atomic model of the functional core binding ATP.

FIG. 3. DNA box nanostructure (black) with functional core. The DNA strands are covalently attached to each other at the verteces although the rendering of the all-atom model suggest seperated strands.

FIG. 4. Examples of modified nucleotides. (A) Adenosine/Lysine-Arginine, (B) Adenosine/Lysine-Lysine, (C) Adenosine/Serine-Glutamic acid, and (D) Cytosine/Histidine.

FIG. 5. (A) Sequence of FlaI of Sulfolobus acidocaldarius (SEQ ID NO:1); (B) amino acid positions of said sequence forming ADP and phosphate binding site; (C) amino acid positions of said sequence forming catalytic center.

FIG. 6. Sequences of functional cores:

FunC1 (SEQ ID NO:2 with amino acid modifications at position 20 (Cytosine bound to Histidine); at position 23 (Adenosine bound to Lysine-Argininge)

FunC2 (SEQ ID NO:3 with amino acid modifications at position 16 (Adenosine bound to Serine-Glutamic Acid); and

FunC3 (SEQ ID NO:4 with amino acid modifications at position 30 (Adenosine bound to Lysine-Arginine).

### DETAILED DESCRIPTION OF THE INVENTION

Specific terms as used throughout the specification have the following meaning.

A “nanostructure” refers to an entity comprising a nucleic acid scaffold and functional core with a catalytic center. The term is not necessarily limited to the nanometer level and may also include entities at the micrometer level having the technical features of the nanostructures as described herein.

A “nucleic acid scaffold” refers to any two-dimensional or three-dimensional structure, object or particle composed of one or more single-stranded nucleic acids, which hybridize to form at least a partially double-stranded structure with defined size and geometry. A nucleic acid scaffold can comprise any of a wide variety of shapes.

As used herein, the terms “nucleic acid molecule”, “nucleic acid”, “polynucleotide”, and “polynucleic acid” are used interchangeably and refer to a polymeric form of nucleotides of any length, either deoxyribonucleotides or ribonucleotides, chemically modified nucleotides or analogs of nucleotides, and combinations of the foregoing. The term includes both, sense and/or anti-sense strands of RNA, synthetic DNA, cDNA, genomic DNA, or a hybrid, where the nucleic acids contain any combination of deoxyribonucleotides, ribonucleotides, single-stranded (ss) and double-stranded (ds) regions and/or any combination of bases, including uracil, adenine, thymine, cytosine, guanine, inosine, xanthine, hypoxanthine, isocytosine, isoguanine, and the like. Nucleic acids may be from natural sources (e.g. genomic, cDNA, RNA), or may be from recombinant or synthetic sources (e.g. produced by chemical synthesis). The term also includes any topological conformation, including single-stranded, double-stranded, partially duplexed, triplexed, hairpinned, circular, and padlocked conformations.

The term “chemically modified nucleotide” refers to nucleotides and/or nucleotide analogs, which differ in their chemical structure from conventional nucleotides and/or nucleotide analogs, having modifications in the chemical structure of the base, sugar and/or phosphate. Nucleotides can be modified at any position on their structure.

Chemically modified bases refer to nucleotide bases such as, for example, adenine, guanine, cytosine, thymine, and uracil, xanthine, hypoxanthine, isocytosine, isoguanine, inosine, and queuosine that have been modified by the replacement or addition of one or more atoms or groups such as 5-position pyrimidine modifications, 8-position purine modifications, modifications at cytosine exocyclic amines, and substitution of 5-bromo-uracil. Exemplary types of nucleotide modifications with respect to the base moieties, include, but are not limited to, alkylated, halogenated, thiolated, aminated, amidated, or acetylated bases, in various combinations as well as bases with one or more bound amino acid residues or amino acid analogs. In case more than one amino acid residues and/or amino acid analog residues are bound to the nucleotide/nucleotide analog, only the first amino acid or amino acid analog residue is covalently bound to the nucleotide/nucleotide analog and the further amino acid or amino acid analog residue(s) are bound to said first residue or any of the further amino acid or amino acid analog residue(s) to form a linear or branched chain of residues.

Chemically modified nucleotides also include nucleotides and/or nucleotide analogs which are modified with respect to the sugar moiety, as well as nucleotides and/or nucleotide analogs having sugars or analogs thereof that are not ribosyl. For example, the sugar moieties may be, or be based on, mannoses, arabinoses, glucopyranoses, galactopyranoses, 4-thioribose, and other sugars, heterocycles, or carbocycles. Modifications of the sugar moiety, e.g. 2′-position sugar modifications, include, but are not limited to, sugar-modified ribonucleotides in which the 2′-OH is replaced by a group such as an H, OR, R, halo, SH, SR, NH₂, NHR, NR₂, or CN, wherein R is an alkyl moiety (i.e., saturated linear or branched hydrocarbon group including, for example, methyl, ethyl, isopropyl, t-butyl, heptyl, dodecyl, octadecyl, amyl, 2-ethylhexyl, and the like). Nucleic acids containing one or more carbocyclic sugars are also included within the definition of nucleic acids.

Chemically modified nucleotides or nucleotide analogs are also meant to include nucleotides with non-natural phosphodiester internucleotide linkages such as methylphosphonates, phosphorothioates, phosphorodithioates, phosphoramides, phosphoramidates, phosphotriesters, in particular alkylesters, phosphoramidites, O-methylphophoroamidite linkages as well as peptide nucleic acid backbones and linkages. Other analog nucleic acids include those with positive backbones, non-ionic backbones and non-ribose backbones. Nucleotide modifications can occur also in changes in the stereochemistry (a-nucleotide phosphodiester) or by attaching different 5′-terminal groups for example such as psoralen and derivatives, phenandroline and derivatives, ellipicitine and derivatives, EDTA, 5′-p(N-2-chloroethyl-N-methylamino)-benzyl-amide, acridine and derivatives.

The term “amino acid” refers to natural amino acids, unnatural amino acids and amino acid analogs, all in their D and L stereoisomers if their structure allows such stereoisomeric forms. Natural amino acids include alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Len), lysine (Lys), methionine (Met), phenylalanine (Phe), praline (Pro), serine (Ser), threonine (Thr), tryptophan (Trp), tyrosine (Tyr) and valine (Val). Unnatural amino acids include, but are not limited to azetidinecarboxylic acid, 2-aminoadipic acid, 3-aminoadipic acid, beta-alanine, aminopropionic acid, Z-aminobutyric acid, 4-aminobutyric acid, 6-aminocaproic acid, 2-aminoheptanoic acid, 2-aminoisobutyric acid, 3-aminoisobutyric acid, 2-aminapirnelic acid, 2,4-diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, alto-isoleucine, N-methylglycine, N-methylisoleucine, N-methylvaline, norvaline, norleucine, ornithine and pipecolic acid.

The term “amino acid analog” refers to natural and unnatural amino acids which are chemically blocked, reversibly or irreversibly, or modified either on the C-terminal carboxy group, the N-terminal, amino group or side-chain functional group to another functional group, as for example, methionine sulfoxide, methionine sulfone, S-carboxymethyl-D-cysteine, S-carboxymethyl-cysteine sulfoxide and S-carboxymethyl-D-cysteine sulfone. For example, aspartic acid-(beta-methyl ester) is an amino acid analog of aspartic acid; N-ethylglycine is an amino acid analog of glycine; or alanine carboxamide is an amino acid analog of alanine.

The term “backbone strand” or “backbone chain” refers to a long nucleic acid sequence, especially a single-stranded nucleic acid sequence, which is capable of assembling into a nucleic acid scaffold by complementary base pairing rules either alone or in combination with staple strands.

The terms “staple strand”, “staple chain”, “oligonucleotide sequence” and “short-chain nucleotide sequence” are used interchangeably herein and refer to nucleic acid sequences, especially single-stranded nucleic acid sequences, which associate at least partially with each other and/or with a backbone strand. Staple chains are capable to assemble with each other into a nucleic acid scaffold by complementary base pairing rules or support assembly of a backbone strand into a nucleic acid scaffold by complementary base pairing rules.

The term “complementarity” or “complementary” as used herein refers to the formation or existence of hydrogen bond(s) between one nucleic acid sequence and another nucleic acid sequence by either traditional Watson-Crick or other non-traditional types of bonding. The term “complementarity/complementary” as used herein includes “reverse complementarity/reverse complementary”. Perfect complementary means that all the contiguous residues of a nucleic acid sequence will hydrogen bond with the same number of contiguous residues in a second nucleic acid sequence. Partial complementarity can include various mismatches or non-based paired nucleotides (e.g. 1, 2, 3, 4, 5, 6, 7, 8, 9, 10 or more mismatches, non-nucleotide linkers, or non-based paired nucleotides) within the nucleic acid molecule, which can result in bulges, loops, or overhangs between the two nucleic acid sequences. Such partial complementarity can be represented by a % complementarity that is determined by the number of non-base paired nucleotides, i.e., about 50%, 60%, 70%, 80%, 90% etc. within the total number of nucleotides involved.

The term “hybridize” or “anneal” refers to the ability of completely or partially complementary nucleic acid strands to come together under specified hybridization conditions in a parallel or preferably antiparallel orientation. The nucleic acid strands interact via hydrogen bonding between bases on opposing strands and form a stable or quasi-stable double stranded helical structure or may result in the formation of a triplex, or other higher-ordered structure. Although hydrogen bonds typically form between adenine and thymine or uracil (A and T or U) or cytosine and guanine (C and G), other base pairs may be formed (e.g., Adams et al., The Biochemistry of the Nucleic Acids, 11th ed., 1992). The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like.

As will be appreciated by persons skilled in the art, stringent conditions are sequence-dependent and are different in different circumstances. For example, longer fragments may require higher hybridization temperatures for specific hybridization than short fragments. Because other factors, such as base composition and length of the complementary strands, presence of organic solvents, and the extent of base mismatching, may affect the stringency of hybridization, the combination of parameters can be more important than the absolute measure of any one parameter alone. In some embodiments, hybridization can be made to occur under high stringency conditions, such as high temperatures or 0.1×SCC. Examples of high stringent conditions are known in the art; see e.g., Sambrook et al., Molecular Cloning: A Laboratory Manual, 2d Edition, 1989, and Short Protocols in Molecular Biology, ed. Ausubel et al. In general, increasing the temperature at which the hybridization is performed increases the stringency. As such, the hybridization reactions described herein can be performed at a different temperature depending on the desired stringency of hybridization. Hybridization temperatures can be as low as or even lower than 5° C., but are typically greater than 22° C., and more typically greater than about 30° C., and even more typically in excess of 37° C. In other embodiments, the stringency of the hybridization can further be altered by the addition or removal of components of the buffered solution. In some embodiments, hybridization is permitted under medium stringency conditions. In other embodiments, hybridization is permitted under low stringency conditions. In some embodiments, a nucleic acid sequence is perfectly complementary to another nucleic acid with which it binds. In other embodiments, one or more mismatches are present between the hybridized molecules or hybridized portions of molecules.

The term “hydrogen bond” as used herein, refers to a form of association between an electronegative atom and a hydrogen atom attached to a second atom exceeding the electronegativity of carbon. The electronegative-atom having a free electron pair to share with the hydrogen atom is the so-called hydrogen bond acceptor, and may be nitrogen, oxygen, sulfur or fluorine. The hydrogen atom bound to the electronegative atom is generally referred to as a hydrogen bond donor. The terms electronegative and electropositive as used herein will be readily understood by the person skilled in the art to mean the tendency of an atom to attract the pair of electrons in a covalent bond so as to lead to an unsymmetrical distribution of electrons and hence the formation of a dipole moment. The hydrogen bond is stronger than a van der Waals interaction, but weaker than covalent or ionic bonds.

The term “covalent bond” or “covalent interaction” refers to bonds or interactions created by the sharing of a pair of electrons between atoms. Covalent bonds/interactions include, but are not limited to atom bonds, homopolar bonds, σ-σ-interactions, σ-π-interactions, two-electron-to-center bonds, single bonds, double bonds, triple bonds, as well as combinations of these interactions/bonds. The mentioned interactions/bonds, can be polar or polarized, or can be non-polar or non-polarized.

“Non-covalent” refers to associations between atoms and molecules such as ionic interactions (e.g. dipole-dipole interactions, ion pairing, and salt formation), hydrogen bonding, non-polar interactions, inclusion complexes, clathration, van der Waals interactions (e.g. pi-pi stacking), and combinations thereof.

As used herein, the term “nanotube” refers to an elongated, hollow nanostructure. In some instances, a nanotube can be represented as comprising an unfilled cylindrical shape. Typically, a nanotube comprises a cross-sectional diameter in the nm range, a length in the μm range, and an aspect ratio that is about 2 or greater.

As used herein, the term “ring” refers to a circular, hollow nanostructure. In some instances, a ring can be represented as comprising an unfilled cylindrical shape. Typically, a ring comprises a cross-sectional diameter in the nm range, a height in the nm range, and an aspect ratio that is about 10 or greater.

As used herein, the term “disc” refers to a circular nanostructure. In some instances, a disc can be represented as comprising a filled cylindrical shape. Typically, a disc comprises a cross-sectional diameter in the nm range, a height in the nm range, and an aspect ratio that is about 10 or greater.

As used herein, “aspect ratio” refers to the ratio of the longest dimension to the shortest dimension of a nanostructure. Therefore, an increase in aspect ratio would indicate that the longest dimension has increased in ratio compared to the shortest dimension.

As used herein the term “catalytic center” refers to amino acid residues of a protein, which are involved in catalyzing a chemical or biological reaction.

As used herein the term “functional core” refers to the part of a nanostructure which is involved in catalyzing a biological or chemical reaction and enables any conformational changes required for this reaction. The functional core, thus, represents an analog of the protein (the native polypeptide) or part of the protein to be emulated within the nucleic acid scaffold. The functional core comprises ordinary and chemically modified nucleotides with one or more amino acid residue(s) and/or one or more amino acid analog(s). The enzymatic reaction is performed by the amino acid residues and/or the amino acid analogs, which form the catalytic center. Thus, the functional core comprises an amino acid based catalytic center analogous to the catalytic center of the protein/native polypeptide to be emulated as well as a nucleic acid based part analogous to the part involved in conformational changes of the protein/native polypeptide.

“Enzymatically active” refers to the ability to measurably catalyze a biological or chemical reaction. Enzymatic activity can be measured by methods and assays known in the art including, but not limited to, methods and assays based on a detectable signal such as chemical or physical signals.

It is an object of the invention to provide a nanostructure comprising a nucleic acid scaffold and a functional core. The nanostructure described herein may be formed into any desired shape depending on the shape of the nucleic acid scaffold it comprises and is usually between 10-5,000 nm in diameter, but larger nanostructures or scaffolds of 10, 15 or 20 μm in diameter may also be used

Provided herein are nanostructures comprising a nucleic acid scaffold and at least one functional core (e.g. a nucleic acid molecule comprising at least one nucleotide or nucleotide analog with one or more amino acid or amino acid analog residue(s) bound to it). In some embodiments, the nanostructure comprises one or more functional core(s), e.g., at least any one of 2, 3, 4, 5 or 6 functional cores. In some embodiments, more than one functional core (e.g. any one of 2, 3, 4, 5 or 6 functional cores) form together one catalytic center. In some embodiments, each functional core of a nanostructure described herein forms a separate catalytic center. In some embodiments, the amino acid or amino acid analogs of the one or more functional core(s) form a catalytic center and further binding sites for substrates, products and/or binding sites for any cofactor(s) of the catalytic reaction. In some embodiments, the nanostructures provided herein comprise a nucleic acid scaffold and one or more functional core(s) forming a catalytic center, wherein the nucleic acid scaffold emulates any structural or functional proteins from where the catalytic center is derived (e.g. the native protein(s)/protein complex performing the respective catalytic activity in nature). The nanostructure may further comprise any of such structural or functional proteins (e.g. proteins of a flagellum or archaellum) or fragments thereof (e.g. FlaX or FlaI or fragments thereof).

A functional core comprises ordinary and chemically modified nucleotides/nucleotide analogs with amino acid side chains or amino acid analogs side chains (e.g. with one or more amino acid residue(s) or amino acid analog residue(s) bound, preferably covalently bound, to a nucleotide or nucleotide analog wherein the first residue is attached (e.g. via a cross-linker) to the nucleotide/nucleotide analog and optionally, further residues are bound to said first or any of the further residue(s) to form a linear or branched chain of residues). For example, a functional core comprises nucleotides/nucleotide analogs wherein at least one nucleotide/nucleotide analog (at least any one of 1,2, 3, 4, 5, 6, 7, 8, 9 or 10 nucleotide(s) or nucleotide analog(s) within the functional core is chemically modified with one or more amino acid or amino acid analog residue(s) (e.g., any one of 1, 2, 3, 4, 5, 6, 7, 8, 9, 10 or 15 residue(s) bound to it).The enzymatic reaction of the nanostructure provided herein is performed by the amino acid residue(s) and/or the amino acid analog residue(s) of the functional core(s). Binding of substrates, cofactors and/or products may occur via further amino acid/amino acid analog residues of the functional core(s) (e.g. residues bound to nucleotides/nucleotide analogs of the one or more functional core(s)). Amino acid residues of the protein (native polypeptide) or part of the protein to be emulated, which are not required for this catalytic reaction per se but for the conformational changes of the protein accompanying such reaction are replaced by the nucleic acid based part of the functional core.

The nucleic acid scaffold of the nanostructure described herein may be “tightened” to prevent ion diffusion. Specifically, the nuclei acid scaffold comprises one or more nucleotides or nucleotide analogs with amino acid side chains (e.g. nucleotides or nucleotide analogs with one or more amino acid residues or amino acid analogues bound (e.g. covalently bound) to any position in the nucleotide). In some embodiments, the amino acid side chains attached (e.g. via cross-linkers) to the nucleotide(s) within a scaffold comprise at least any one of 1, 2, 3, 4, 5, 6, 7, 8, 9, 10, 15, 20, 30, 40, or 50 amino acid residues or amino acid analogues, wherein the first amino acid or amino acid analog residue is bound (e.g. via a cross-linker) to the nucleotide/nucleotide analog and optionally, any further amino acid or amino acid analog residue(s) are bound either directly to the first amino acid or amino acid analog residue or indirectly (via the second, third etc. residue), forming a linear or branched chain of amino acid or amino acid analog residues. The distance of the amino acids or amino acid analogs of different side chains within the scaffold may be less than 100 pm. Such tightening of one or more scaffold(s) can also be achieved by addition of intercalating peptides that bind via hydrogen bonds to the nucleic acid scaffold(s) and create a tight coating.

Chemical modification of the nucleotide/nucleotide analogs with amino acid/amino acid analogs may be as follows: For example, any one of 1, 2, 3, 4, 5, 6, 7, 8, 9, 10, 11, 12, 13, 14, or 15 amino acid or amino acid analog residues may be bound to the nucleotide/nucleotide analog. In some instances, any one of 1 to 5, 5 to 15, 10 to 30 or even up to 50 amino acid or amino acid analog residues may be bound. If more than one residue is bound, the amino acid residues are bound as dipeptide or a linear or branched oligopeptide or polypeptide via the first residue. In some embodiments, the first amino acid or amino acid analog residue of such chemically modified nucleotide is bound via a cross-linker.

In order to introduce chemical modification “click chemistry” may be used. In general terms, “click chemistry” describes reactions used to join small chemical subunits in a modular fashion, yielding singular reaction products that are typically physiologically stable and stereospecific. Click chemistry applications make use of azide alkyne Huisgen cycloaddition, a two-step process that uses quantitative chemical reactions of alkyne and azide moieties to create covalent carbon-heteroatom bonds between biochemical species.

Other typical cross-linkers or attachment chemistries used for attaching a molecule (e.g. an amino acid/amino acid analog) to a nucleic acid molecules include but are not limited to biotins, (e.g. Biotin dT, Biotin-TEG), amino modifiers (e.g. 5′ Amino Modifier C6, 5′ Amino Modifier C12, Amino Modifier dT, Uni-Link™ Amino Modifier), azide (NHS esters), alkynes (e.g. 5′Hexenyl, 5′Octadinynyl dU), and thiol modifiers (e.g. dithiol, dithiol phsphoramidite (DPTA)). In some embodiments, the cross linker is an azide/NHS ester. In some embodiments, the cross linker is an alkyne, e.g. 5-Octadinynyl dU.

Biotins are frequently used in attachment chemistry. 5′ Biotin is a versatile linker. 5′ Dual Biotin inserts two adjacent biotin moieties in a sequence, which can slightly increase affinity to streptavidin. Biotin dT allows placement of a biotin internally without disrupting nucleotide spacing. Biotin-TEG helps reduce steric hindrance in applications that require the use of magnetic beads.

Amino Modifiers provide an alternative attachment chemistry. Primary amines are reactive with a number of useful molecules such as isothiocyanates, NHS esters, or activated carboxylates. The amino-modifier 5′ Amino Modifier C6 with a spacer arm of 6-7 atoms is the simplest choice. 5′ Amino Modifier C12 increases the distance between the functional amine and the DNA sequence. Amino Modifier dT inserts the functionality internally from an added dT base while the Uni-Link™ Amino Modifier does so without an additional nucleotide.

Azide (NHS Ester) modifications use an NHS ester functional group to attach an azide moiety at the 5′, 3′, or any internal position in an oligonucleotide. This azide moiety may subsequently be used to attach alkyne modified groups using the click reaction.

Alkyne modifiers are used to react with azide-labeled functional groups to form stable bonds through the click reaction. 5′ Hexynyl is the simplest and most popular way to introduce a 5′ terminal alkyne group. 5-Octadinynyl dU is a modified base with an 8-carbon linker terminating in an alkyne group and is the preferred way to insert alkynes at internal positions within a sequence, but can also be used for 3′ or 5′ attachment.

A thiol group can be used to attach an oligonucleotide to a variety of fluorescent and nonfluorescent moieties or surfaces. Dithiol can be inserted into an oligonucleotide at the 5′ position, the 3′ position or internally. Each insertion results in two SH groups available for coupling with ligands or surfaces. The dithiol phosphoramidite (DTPA) modification can be inserted in series so that 2, or even 3, groups can be positioned adjacent to each other to increase efficiency of ligand/surface interactions.

The one or more functional core(s) may be bound covalently or via staple oligonucleotides (e.g. via hydrogen bonds between complementary sequences) to the nucleic acid scaffold or through affinity interaction such as e.g. biotin-avidin/strepavidin interaction, or by any other anchoring approach. In some embodiments, the functional core(s) are directly bound to the nucleic acid scaffold by a covalent bond or via staple oligonucleotides. For example, staple oligonucleotide(s) involved in the assembly/formation of the nanostructure may further act as functional core(s) by providing at least one nucleotide/nucleotide analog with one or more amino acid/amino acid analog residue(s) forming the catalytic center.

In some embodiments, the functional core(s) are indirectly bound or attached to the nucleic acid scaffold. Specifically, the functional core(s) may be indirectly bound or anchored to the nucleic acid scaffold via molecules that may serve as anchors or cross-linkers. For example, molecules that may serve as anchors or cross-linkers for binding other known specific molecules include, but are not limited to antibodies, ferritin, polyhistidine tag, c-myc tag, histidine-tag, hemagglutinin tag, biotin, avidin, streptavidin and the like.

The nanostructure described herein may be two-dimensional (2D) or three-dimensional (3D). Specifically, the nanostructures may form a molecular structure or object defining an interior space. For example, the nucleic acid scaffold of the nanostructure may have the shape of a hollow structure or object, such as a nanotube, cylinder, ring, box, a pyramide, a cross or cube comprising an interior space. The one or more functional core(s) thus may be located or embedded within the interior space of such hollow nucleic acid scaffold.

In some embodiments, the nucleic acid scaffold of the nanostructures described herein is a nanotube, which comprises one or more functional core(s) within its interior/hollow space (e.g. one or more functional core(s) embedded in the interior space of the nanotube). In some embodiments, the nucleic acid scaffold of the nanostructures described herein is a box, a pyramide or a cylinder. In some embodiments, the nucleic acid scaffold is a box, a cylinder or a pyramide comprising one or more functional core(s) within its interior/hollow space.

In some embodiments, the nanostructure described herein is a molecular motor comprising a nucleic acid scaffold forming a hollow nanotube, cylinder, pyramide or box with one or more functional core(s) (e.g. any one from 2, 3, 4, 5 or 6 functional cores) comprising the catalytic center of an ATPase (e.g. FlaI) located in its hollow interior space, wherein the nanotube, cylinder, pyramide or box emulates one or more structural or functional proteins of a flagellum or archaellum (e.g. any one or more of protein FlaI, FlaX, FlaH and FlaJ) or fragments thereof, which are required as anchoring structures, activity regulation or force transmission.

In some embodiments, the nanostructure described herein is composed of a first nucleic acid scaffold composed of at least two substructures (e.g. two rings or discs) with a hollow interior space and with one or more functional cores comprising the catalytic center of an ATPase (e.g. FlaI), and optionally binding sites for substrate(s) or cofactor(s), located in said interior space, wherein the substructures emulate one or more structural or functional proteins of a flagellum or archaellum (e.g. any one or more of protein FlaI, FlaX, FlaH and FlaJ) or fragments thereof, which are required as anchoring structures, for activity regulation or force transmission. In some embodiments, the molecular motor comprises one or two rings or discs with a hollow interior space formed by a nucleic acid scaffold with one or more functional core(s) (e.g. any one of 3, 4, 5, or 6 functional cores) comprising the catalytic center of an ATPase (e.g. FlaI) located in said interior space, wherein the substructures emulate a structural or functional proteins of a flagellum or archaellum (e.g., FlaX of a archaeal rotary motor) or fragments thereof. Thus, the assembled molecular motor (e.g. molecular motor with at least two substructures) comprises rotating, moving and anchored substructures and at least one functional core. In some embodiments, the molecular motor further comprises one or more structural proteins of an archaellum or flagellum (e.g. an archeal rotary motor) or fragments thereof (e.g. FlaX, FlaH, FlaJ and/or FlaI or fragments thereof).

Nucleic Acid Scaffold

The nucleic acid scaffold can be fabricated from one or more nucleic acid molecule(s). Nucleic acid nanotechnology makes use of the fact that, due to the specificity of Watson-Crick base pairing, only portions of the strands which are complementary to each other will bind to each other to form a duplex. Construction of nucleic acid scaffolds or nanostructures has been described in several publications, including WO 2008/039254, US 2010/0216978, WO 2010/148085, U.S. Pat. No. 5,468,851, U.S. Pat. No. 7,842,793, Dietz et al. (2009) [Dietz et al., Science 325:725-730(2009)], Douglas et al. (2009) [Douglas et al., Nature 459:414 (2009)]. Essentially, natural or artificial nucleic acid sequences can be programmed to generate structures, objects or particles of defined size and geometry. Usually, DNA-based scaffolds make use of a single strand of DNA (backbone chain), which is induced into a specific conformation by the binding of complementary, shorter DNA strands (staple chains). Scaffolds based on folded single-stranded DNA are also feasible, for example, via self-hybridizing segments of one long single-stranded DNA, as well as scaffolds assembled by a plurality of staple chains or oligonucleotides without a long (backbone) strand. RNA typically folds into specific structures by forming tertiary RNA motifs, based on RNA-RNA interactions within the same molecule. Alternatively, RNA structures may be assembled by RNA duplexes.

For creating shapes by folding a backbone chain into a desired shape or structure using a number of small staple chains as glue to hold the scaffold in place, the number of such helper or staple strands will depend upon the size of the backbone strand and the complexity of the shape or structure. For example, for relatively short backbone strands (e.g. about 150 to 1,500 base in length) and/or simple structures the number of helper/staple strands may be small (e.g. about 5, 10, 50 or more). For longer backbone strands (e.g. greater than 1,500 bases) and/or more complex structures, the number of helper strands may be several hundred to thousands (e.g. 50, 100, 300, 600, 1,000 or more helper strands). The choice of staple strands determines the pattern. A software program may be used to identify the staple strands needed to form a given design. Popular programs to design DNA nanostructures including the automated design of helper/staple strands are cadnano [Douglas et al., NAR 37(15):5001-6 (2009)], vHelix [Benson et al., Nature 523:441 (2015)] and Daedalus [Veneziano et al., Science 352:1534 (2016)].

The backbone chain may be a circular or linear nucleic acid. In some embodiments, the backbone strand comprises at least any one of 150, 300, 500, 750, 1,000, 1,250 or at least 1,500 nucleotides. In some embodiments, the backbone strand comprises more than 1,000 nucleotides. In some embodiments, the nucleic acid scaffold comprises at least any one of 5, 10, 20, 30, 40, 50, 100, 300, 500, 800 or 1,000 staple strands. In some embodiments, the scaffold is formed or comprises more than 50 staple strands. In some embodiments, the staple strand comprises at least 30, 40, 50, 60, 70, 80, 90 or 100 nucleotides. In some embodiments, the staple strand comprises more than 30 nucleotides. In some embodiments, the staple strand may be less than 500, less 400, less than 300, less than 200, less than 100, or less than 50 nucleotides in length. In some embodiments, the staple strands are at least any one of 90%, 95% or 100% complementary to each other and/or to a backbone strand.

In some embodiments, the scaffold is formed in a self-assembly process, for example, staple chains hybridize to a backbone strand to complete the formation of self-assembled structure by nucleic acid complementary base pairing rules. In some embodiments, the nucleic acid scaffold is assembled by DNA origami. DNA origami is a method of generating DNA artificially folded at nano scale, creating an arbitrary two or three dimensional shape that may be used as a scaffold for trapping inside, or capturing, an entity. Methods of producing DNA scaffolds of the origami type have been described, for example, in U.S. Pat. No. 7,842,793. DNA origami involves the folding of a long single strand of DNA (e.g. viral DNA) aided by multiple smaller “staple” strands. These shorter strands bind the longer strand in various places, resulting in the formation of a 2D or 3D structure.

Nucleic acid scaffolds as described herein may be composed of deoxyribonucleotides or ribonucleotides, chemically modified nucleotides or analogs of nucleotides, and combinations of the foregoing.

As will be appreciated by those in the art, any nucleic acid analogs and/or chemically modified nucleotides/nucleotide analogs described herein may (e.g.

nucleotides/nucleotide analogs with one or more amino acid/amino acid analog residues attached, optionally for tightening the nucleic acid scaffold) find use as helper or staple strands (staple chains) or as part of a polynucleotide or backbone chain used to generate the nucleic acid scaffold. In addition, mixtures of naturally occurring nucleic acids and analogs can be used. For example, PNA (Peptide nucleic acids) includes peptide nucleic acid analogs, which have increased stability. Thus, nucleic acids of various forms and conformations may be used for generating the nucleic acid scaffold, including right-handed DNA, right-handed RNA, PNA, locked nucleic acid (LNA), threose nucleic acid (TNA), glycol nucleic acid (GNA), bridged nucleic acid (BNA), phosphorodiamidate morpholino oligo (PMO), as well as nucleotide analogues, such as non-Watson-Crick nucleotides dX, dK, ddX, ddK, dP, dZ, ddP, ddZ.

In some embodiments, the nucleic acid scaffold is a DNA scaffold. In some embodiments, the nucleic acid scaffold is a RNA scaffold. In some embodiments, the nucleic acid scaffold is composed of both, DNA and RNA. In some embodiments, the nucleic acid scaffold comprises one or more chemically modified nucleotides or nucleotide analogues. The nucleic scaffold may comprise DNA:DNA duplexes, DNA:RNA, RNA:RNA, DNA:PNA duplexes or any combination thereof.

In some embodiments, the nucleic acid scaffold is composed of a single backbone strand (e.g. a single-stranded DNA or RNA backbone strand). In some embodiments, the nucleic acid scaffold is composed of one backbone strand (e.g. a single-stranded DNA or RNA backbone strand) and a plurality of staple strands (e.g. at least 50 single-stranded RNA or DNA staple strands comprising at least 30 nucleotides). In some embodiments, the nucleic acid scaffold is composed of a plurality of staple strands or oligonucleotides (e.g. at least 50 single-stranded DNA or RNA oligonucleotides comprising at least 30 nucleotides). In some embodiments, the scaffold comprises staple strands of the same length (e.g. each strand comprising at least 30 nucleotides). In some embodiments, the scaffold is formed or comprises staple strands of a plurality of lengths (e.g. 5-10 staple strands comprising at least 30 nucleotides, and 5-10 staple strands comprising at least 50 nucleotides).

A backbone chain or strand may be a M13 phage genomic DNA, Lambda phage genomic DNA or an artificial DNA fragment. Isothermal amplification can be used to generate long DNA fragments also out of any short, circular DNA template.

A nucleic acid scaffold may form any 2D or 3D structure, object or particle. Typical nucleic acid scaffolds have a spatial resolution of about 5 nm to about 500 nm, though the spatial resolution may be greater than 500 nm. Examples of 2D or 3D shapes formed by the nucleic acid scaffold include but are not limited to a sheet, square, rectangle, nanotube, cylinder, ring, disc, ribbon, box, cube, pyramide and rod. In some embodiments, the nanostructure described herein comprises a nucleic acid scaffold forming a box, cylinder or pyramide. In some embodiments, the nanostructure comprises a nucleic acid scaffold forming a box, cylinder or pyramide, any of which having an interior hollow space, and one or more functional cores providing a catalytic center(s) and optionally binding site(s) for any substrates/cofactors within the interior (hollow) space of the box, cylinder or pyramide.

Functional Core

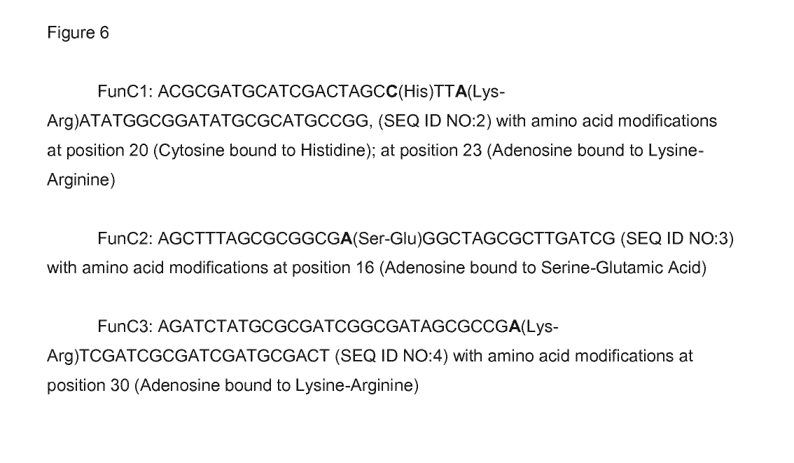

The nanostructure described herein comprises at least one functional core (e.g. any one of 1, 2, 3, 4, 5 or 6 functional cores which may act together to catalyze an enzymatic reaction or which act independent of each other to catalyze different enzymatic reactions). The functional core comprises nucleotides, nucleotide analogs, amino acids and/or amino acid analogs (e.g. staple chains with one or more modified nucleotides or nucleotide analogs) within the nanostructure that enable conformational changes during the catalytic activity of the nanostructure as described herein as well as the catalytic activity itself, and optionally for binding of substates and/or cofactors, and any conformational changes associated with such (catalytic) activity of the nanostructure. The functional core may be composed of one or more nucleic acid strands (e.g. staple strands), each comprising at least 10, 15, 20, or 30 nucleotides/nucleotide analogs and amino acids and/or amino acid analogs. Specifically, the functional core comprises at least one chemically modified nucleotide, preferably a nucleotide comprising one or more amino acid or amino acid analog residue(s) which form a catalytic center (e.g., a nucleotide or nucleotide analog with any one of 1, 2, 3, 4, 5, 6, 7, 8, 9, 10, or 15, amino acid residues and/or amino acid analogs, wherein the first amino acid residue or amino acid analog residue is bound to the nucleotide and any further residues are bound either directly to the first amino acid or amino acid analog residue or indirectly (via the second, third etc. residue), forming a linear or branched chain of amino acid or amino acid analog residues). In some embodiments, the functional core comprises at least one nucleotide with a polypeptide side chain (e.g. any one of 1 to 5 nucleotides/nucleotide analogs). In some embodiments, at least 5%, 10%, 20%, 30%, 40%, 50%, 60%, 70%, 80% or 90% of the nucleotides of the functional core are chemically modified (e.g. comprise one or more amino acid residues and/ or amino acid analogs). In some embodiments, the functional core comprises the sequence of any one of SEQ ID NO:2 to 4 with chemical modifications as shown in FIG. 6.

The catalytic center formed by one or more functional cores may comprise at least 5, 6, 7, 8, 9, 10, or 15 amino acid residues and/or amino acid analogs, preferably from 5-15 or from 5-10 amino acid or amino acid analog residues. The amino acid residues and/or amino acid analogs forming the catalytic center may be derived from (e.g. bound to) the same or different nucleotides or nucleotide analogs of a functional core. For example, one or more amino acids or amino acid analogs bound to nucleotide X, and one or more amino acid residues and or amino acid analogs bound to nucleotide Y may form together the catalytic center, wherein nucleotide X and Y may be within the same or different functional core(s). In some embodiments, more than one functional core (e.g. any one of 2, 3, 4, 5 or 6 functional cores) form together one catalytic center. In some embodiments, each functional core of the nanostructure described herein forms a separate catalytic center. In some embodiments, the amino acid or amino acid analogs of the one or more functional core(s) form one or more catalytic center(s) and further binding sites for substrates, products and/or binding sites for any cofactor(s) of the catalytic reaction.

Functional cores may be designed by a software program calculating the movement of the 4D model of a nanostructure as described herein in in a user-defined area (e.g. the conformational changes of a catalytic center upon hydrolysis of a substrate). For example, WHAT IF Software (Vriend, Journal Mol Graph., 8,52-56 (1990)) and AMBER (Meagher et al., Journal of Computational Chemistry, 24:1016-25 (2003); Allner et al., J. Chem. Theory Comput:, 8(4):1493-1502 (2012)) may be employed to optimize the geometry of the nanostructure and/or functional core based on crystal structures of proteins with catalytic activity (e.g. ATPases). The atoms which are essential for the reaction of interest including atoms that are not directly involved in the chemical reaction but required for conformational changes are selected and define the functional core.

The sequences of the nucleic acid molecules with chemical modifications (e.g. one or more bound amino acid residues), are tested regarding the steric effects of such modifications in software programs known in the art (e.g. MAESTRO/SCHRÖDINGER, (Schrödinger Release 2016-3: M S Jaguar, Schrödinger, LLC, New York, N.Y., 2016)). Further testing of the nucleic acid molecules with chemical modifications regarding their ability to form/emulate catalytic centers of known proteins based on their crystal structure can be performed using, for example, Visual Molecular Dynamics (VMD, Humphrey et al., Journal Mol. Graph, 14:33-38, (1996)), and MAESTRO/SCHRÖDINGER (Schrödinger Release 2016-3: M S Jaguar, Schrödinger, LLC, New York, N.Y., 2016).

The nucleotides or nucleotide analogs of the functional core (e.g. staple chains with one or more modified nucleotides and/or nucleotide analogs) are directly or indirectly associated with the nucleic acid scaffold and may be part of the general structure formed by the nucleic acid scaffold (e.g. a cylinder, pyramide, box, nanotube, ring, disc or the like) or may form a substructure of any shape (e.g. cylinder, pyramide, box, nanotube, ring, disc, or the like). For example, the nucleic acid scaffold of the nanostructure may form a nanotube, cylinder, pyramide or box and the functional core may form a ring or disc (e.g. by hybridization of one or more staple strands having chemically modified nucleotides), which is bound or associated to the nucleic acid scaffold (e.g. a ring or disc having the same diameter or different diameter as the nucleic acid scaffold). Alternatively, the nucleotides or nucleotide analogs of the functional core (e.g. staple chains having one or more modified nucleotides or nucleotide analogs) may hybridize with the backbone strand and/or staple strands forming the nucleic scaffold and contribute to the formation of the nucleic acid scaffold and nanostructure (e.g. a cylinder, pyramide, box, nanotube, disc or ring).

Catalytic Center

The catalytic center of the nanostructures described herein enables a chemical or biological reaction. For example, the catalytic center is derived from an ATP-driven motor (e.g. amino acid residues of the catalytic center of an ATP-driven motor). Such ATP-driven motors hydrolyze ATP to generate chemical free energy which they use to perform mechanical work. Molecular motors based on arrays of motor proteins capable of moving one array and its attached substrate using ATP are disclosed in WO01/09181. Specifically, catalytic centers of rotary motors are used in the nanostructures described herein.

For example, the F₀F₁-ATP synthase family of proteins converts the chemical energy in ATP to the electrochemical potential energy of a proton gradient across a membrane or the other way around. The catalysis of the chemical reaction and the movement of protons are coupled to each other via the mechanical rotation of parts of the complex. This is involved in ATP synthesis in the mitochondria and chloroplasts as well as in pumping of protons across the vacuolar membrane. The bacterial flagellum responsible for the swimming and tumbling of E. coli and other bacteria acts as a rigid propeller that is also powered by a rotary motor. This motor is driven by the flow of protons across a membrane, possibly using a similar mechanism to that found in the F₀ motor in ATP synthase. The archaellum of archaea is a type VI pilus-like structure of archaea, which confers motility by rotary movement of the filament. The archaellum consists of different proteins including an ATPase, FlaI, and FlaX, which forms an oligomeric ring structure.

Specifically, the nanostructures described herein comprise the catalytic center of the archaeal rotary motor (e.g. the catalytic center of the ATPase FlaI). The archaeal motor is present in the large majority of motile archea. The catalytice center of the nanostructures described herein may be derived from species including but not limited to Acidilobus saccharovorans, Aeropyrum pernix, Archaeoglobus fulgidus, Halobacterium salinarum, Haloferax volcanii, Metallosphaera sedula, Methanococcus maripaludis, Methanococcus voltae, Nitrosoarchaeum limnia, Nitrososphaera gargensis, Sulfolobus acidocaldarius, or Thermosphaera aggregans. Although the proteins of the archaellum share sequence similitarity throughout the archaeal kingdoms, the protein complex differs between the different species. The archaella operon of Acidilobus saccharovorans comprises FlaB, FlaG, FlaH, FlaI and FlaJ (sequences available under accession number NC_014374). The archaella operon of Aeropyrum pernix comprises FlaB, FlaX, FlaG, FlaF, FlaH, FlaI and FlaJ (sequences available under accession number BA000002.3). The archaella operon of Archaeoglobus fulgidus comprises FlaB1-2, FlaB1-1, FlaD/E, FlaG, FlaF, FlaH, FlaI and FlaJ (sequences available under accession number CP006577.1). The archaella operon of Halobacterium salinarum comprises FlaA1, FlaA2, FlaB1, FlaB2, FlaB3, FlaC/D/E, FlaD, FlaG, FlaF, FlaH, FlaI, FlaJ and FlaK (sequences available under accession number NC_010366.1). The archaella operon of Haloferax volcanii comprises FlaA1, FlaA2, FlaB1, FlaC/E, FlaF, FlaG, FlaH, FlaI and FlaJ (sequences available under accession number NC_013967.1). The archaella operon of Metallosphaera sedula comprises FlaB, FlaX, FlaG, FlaF, FlaH, FlaI and FlaJ (sequences available under accession number NZ_CP012176.1). The archaella operon of Methanococcus maripaludis comprises FlaB1, FlaB2, FlaB3, FlaC, FlaD, FlaE, FlaF, FlaG, FlaH, FlaI and FlaJ (sequences available under accession number NC_009975.1). The archaella operon of Methanococcus voltae comprises FlaA, FlaB1, FlaB2, FlaB3, FlaC, FlaD, FlaE, FlaF, FlaG, FlaH, FlaI and FlaJ (sequences available under accession number NC_014222.1). The archaella operon of Nitrosoarchaeum limnia comprises FlaB1, FlaB2, FlaB3, FlaB4, FlaF, FlaG, FlaH, FlaI and FlaJ (sequences available under accession number CM001158.1). The archaella operon of Nitrososphaera gargensis comprises FlaB, FlaF, FlaG, FlaH, FlaI and FlaJ (sequences available under accession number CP002408.1). The archaella operon of Sulfolobus acidocaldarius comprises FlaB, FlaX, FlaF, FlaG, FlaH, FlaI and FlaJ (sequences available under accession number NC_007181.1). The archaella operon of Thermosphaera aggregans comprises FlaB, FlaG, FlaH, FlaI and FlaJ (sequences available under accession number CP001939.1). The catalytical center of the archaeal rotary motor is embedded in the FlaI protein which was demonstrated to have ATP hydrolyzing activity. In general, FlaI forms an ATP-dependent hexamer with Walker A and Walker B motifs for ATP-binding and hydrolysis. For more details see Reindl et al. (Mol. Cell., 49(6):1069-82 (2013)) and Ghosh et al. (Biochem J., 437(1):43-52 (2011)).

The nanostructures described herein further encompass nanostructures with motor activity, structural functions and/or actual movement. In some embodiments, the nanostructure is composed of a nucleic acid scaffold, which comprises at least two substructures such as at least two rings or discs with different diameter but having the same geometric center or a geometric center positioned on the same axis, and at least one functional core. These substructures are nucleic acid scaffold analogs of the e.g.

archaeal rotary motor components FlaI, FlaX, FlaH and FlaJ (e.g of Sulfolobus acidocaldarius). The substructures comprise analogous elements of these proteins essential for the correct function such as a functional core comprising the catalytical center, anchoring structures, activity regulation sites and force transmission elements. Thus, the assembled nanostructure comprises catalytically active, rotating, moving and anchored substructures.

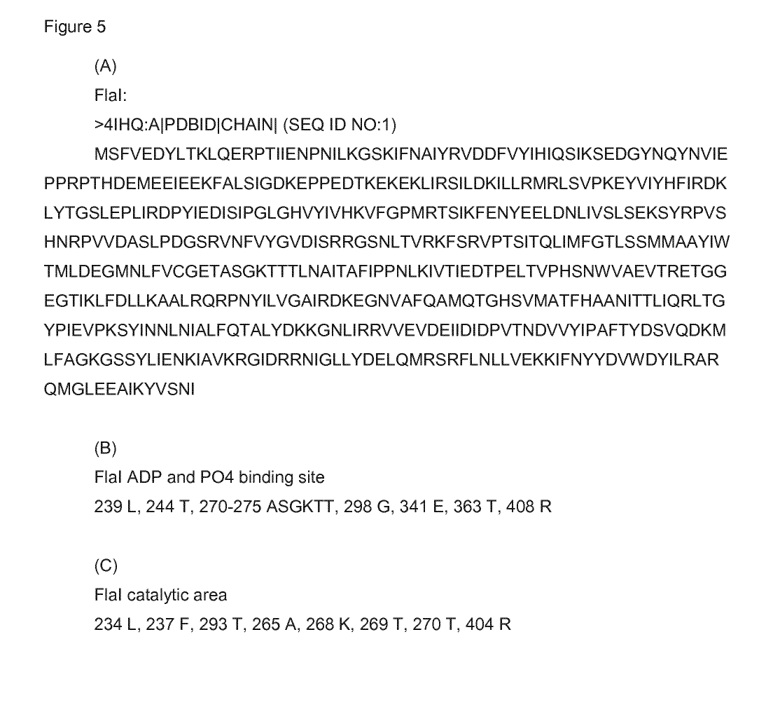

In some embodiments, the nanostructure described herein is composed of a nucleic acid scaffold with one or more functional core(s) comprising a catalytic center of an ATP-driven motor embedded therein and optionally one or more structural or functional nucleic acid-based protein analogs of a flagellum or archaellum or fragments of such proteins. In some embodiments, the one or more protein analogs are bound to the nucleic acid scaffold. In some embodiments, the protein analog emulates an ATPase and/or a structural protein involved in forming a ring or filament required for movement of the flagellum or archaellum it is derived from. In some embodiments, the protein analog emulates the ATPase FlaI (e.g. FlaI comprising the sequence of SEQ ID NO:1; FIG. 5) and/or FlaX of an archaellum (e.g. of Sulfolobus acidocaldarius). In some embodiments, the nanostructure described herein is composed of a nucleic acid scaffold (e.g. forming the shape of a hollow box, cylinder, pyramide or ring) and one or more functional core(s) comprising a nucleic acid molecule with at least one chemically modified nucleotide (e.g. the nucleic acid molecules FunC1 (SEQ ID NO:2); FunC2 (SEQ ID NO:3) and/or FunC3 (SEQ ID NO:4) with chemical modificatios as shown in FIG. 6), which forms a catalytic center of an ATPase and enabling any binding of substrates and/or cofactors and/or conformational changes associated with such catalytic activity of the nanostructure. Specifically, the nanostructure described herein is composed of a nucleic acid scaffold (e.g. forming the shape of a hollow box, cylinder, pyramide or ring) and at least three functional cores, e.g. the functional cores of SEQ ID NO:2-4, wherein SEQ ID NO:2 comprises amino acid modifications at position 20 (Cytosine bound to Histidine); and at position 23 (Adenosine bound to Lysine-Argininge), SEQ ID NO:3 comprises amino acid modifications at position 16 (Adenosine bound to Serine-Glutamic Acid), and SEQ ID NO:4 comprises amino acid modifications at position 30 (Adenosine bound to Lysine-Arginine).

The catalytic center of the nanostructures described herein may be a catalytic center of an enzyme preferably of an ion pump, a light harvesting complex or a photosystem.

An ion transporter or ion pump, is a transmembrane protein that moves ions across a plasma membrane against their concentration gradient. These primary transporters are enzymes that convert energy from various sources, including ATP, sunlight, and other redox reactions, to potential energy stored in an electrochemical gradient. This energy is then used by secondary transporters, including ion carriers and ion channels, to drive vital cellular processes, such as ATP synthesis. Thus, in some embodiments, the catalytic center is derived from an ATPase of an ion pump. In some embodiments, the catalytic center is derived from an enzyme involved in redox reactions.

### EXAMPLES

### Example 1

### Generation of Nucleic Acid Scaffold

The generation of a functional nanostructure requires an atomistic and time-resolved 3D (4D) model of the biological structure that is going to be emulated. This 4D model is used as input data for a computer program. The program calculates the movement of the atoms in the 4D model in a user-defined area (e.g. the conformational changes of a catalytic center upon hydrolysis of a substrate). The atoms are selected that are essential for the reaction of interest including atoms that are not directly involved in the chemical reaction but required for conformational changes. In general, the selected atoms that are part of the catalytical center and/or involved in conformational changes are integrated by the program into a nucleic acid scaffold in a manner that they spatial position remains unchanged relative to each other.

The DNA scaffold is generated by methods summarized under the term “DNA origami” and comprises long and short single-stranded nucleic acid strands. Specifically, the workflow starts with the design of the multilayer target shape and the determination of the staple sequences using a computer program (e.g. caDNAno). In the next step, the genomic DNA of the e.g. M13mp18 bacteriophage can be used as long single-stranded nucleic acid strand. Other possibilities to obtain the long single-stranded nucleic acid strand include e.g. enzymatic digestion of one strand of a double-stranded plasmid or separation of PCR amplicons. Short single strands are in general obtained by chemical synthesis and purification and offered by many commercial vendors. Subsequently, equal amounts of concentration-normalized staple strands (e.g. 500 nM) of each substructure are pooled. The long staple strand can be 2 times or less concentrated (e.g. 100 nM) than the short staple strands. The molecular self-assembly reaction takes place in an aqueous buffer comprising additional ions such as Mg, Cl, K, Na, SO₄, etc. Repeated heating and cooling of the reaction mixture can be used to facilitate the folding reaction. In a nanostructure comprising multiple substructures, the finalized substructures are pooled and the final self-assembly reaction takes place in an aqueous buffer. The analysis of the folding quality can be analysed using TEM or cryo-electron tomography. For this purpose, agarose gel electrophoresis is used to purifiy and excise the desired structures from the gel. A detailed description of this procedure can be found under the reference (Castro et al., Nat Methods, 3:221-9 (2011)).

Nucleic acid strands that are associated with or form the functional core have chemically modified nucleotides (e.g. polypeptide chains). The program generates a file comprising single-stranded nucleic acid strands including appropriate chemical modifications as output.

The single-stranded nucleic acid strands are synthetized and chemically modified, if modifications are appropriate, by commercial suppliers. Subsequently, they are pooled in an aqueous buffer and self-assemble into the programmed structure.

### Example 2

### Generation of Nanostructure with Catalytic Center

The generation of the functional core requires a detailed model on the catalytical center of a native protein (e.g. FlaI). The model includes spatial coordinates of the involved catalytical center (CT) atoms and details on the chemical and conformational changes that are performed during and after the hydrolyzis of a substrate. This information is essential for the computer-aided design of the functional core. Based on this information, the program using e.g. atomistic molecular dynamics simulation calculates staple strands that are covalently attached to the involved CT-atoms and that hybridize within the nucleic acid scaffold in a manner that the original spatial distribution of the CT-atoms remains intact. This process results in chemically modified staple strands with e.g. peptide chain modifications. The sum of all chemically modified staple strands and, more specifically, the chemical modifications of the staple strands reconstitute the native catalytical center. The staple strands are essential to precisely assemble the multiple e.g. peptide chains in space. Thus, the enzymatic activity of the native protein (e.g. FlaI) can be emulated. The functional core generated using this process is integrated into the complete nucleic acid scaffold structure. The generation and assembly of both the functional core and the nucleic acid scaffold is explained above in Example one.

Molecular Modelling of FlaI-FlaI Systems Preparation

Crystal structure of FlaI hexamer containing ADP or ATP as well as PO4 in the catalytic sites (PDB: 4IHQ) was used for building both monomeric and hexameric protein complex models. Water molecules found in the crystal structure within 0.3 nm from the protein heavy atoms were retained for calculation. Polar hydrogen atoms were added using WHAT IF software, and non-polar with the tleap module of the AMBER 16 program package. For the parametrization of protein atoms AMBER ff99SB force field was used. Parameters for ADP, ATP, PO4 and Mg ions were obtained using AMBER parameter database, University of Manchester (Meagher et al., Journal of Computational Chemistry, 24:1016-25 (2003); Allner et al., J. Chem.Theory Comput:, 8(4):1493-1502 (2012))

The FlaI monomer and hexamer were centered in the rectangular parallelepiped box filled with TIP3P water molecules. Na⁺ and Cl⁻ ions were added in order to neutralize the system. Using the described procedure, a total of four systems were prepared, two smaller monomeric systems containing app. 85,000 atoms, and two bigger systems containing app. 290,000 atoms. The difference between same sized systems was only in the substrate bonded to the catalytic site, in one ADP with PO4 and ATP in other.

Simulations and Geometry Optimizations

All systems were energy minimized and their geometry optimized in a process consisting of 50,000 steps of the steepest descent algorithm. The constraint of 418.4 Kj was applied on the protein complex atoms, while all of the solvent molecules remained unconstrained.

After geometry optimization, systems were subjected to molecular dynamics (MD) simulations. FlaI monomer systems were simulated to a maximum of 80 ns, while FlaI hexamer systems to a maximum of 200 ns. The temperature was linearly increased from 0 to 300 K using a Berendsen thermostat. During the first 300 ps, the protein complex atoms were constrained with a force constant of 104.6 Kj and the volume was kept constant.

From 300 ps to the end of simulations, no constraints were applied and simulations were conducted at a constant temperature (300K) and a constant pressure (101 325 Pa) using Berendsen thermostat and barostat. The time step was 2 fs, and the structures were sampled every 10 picosecond. Periodic boundary conditions (PBC) were applied. Particle mesh Ewald (PME) was used for the calculation of electrostatic interactions. The cut-off value for non-bonded interactions was set to 1 nm. All simulations were performed using the AMBER 14 and GROMACS 5.1.4 simulation packages. All trajectories were analyzed using the VMD, GROMACS and SCHRÖDINGER analyzing tools.

Root-mean-square deviation and the radius of gyration calculations were used to inspect general system stability and to evaluate need for prolongation of MD simulations.

ATP Binding Site Emulation

The catalytic center screening was made with the purpose of detecting similarity in amino acids, and to investigate the common interaction principle between ATP/ADP and the catalytic sites in several different protein complexes (PDB: 4IHQ, 3PUW, 2OAP, 3PUV, 3RLF). Screening was performed by visual inspection using VMD and Maestro from the Schrödinger program package.

The analysis was based on crystal structures of protein complexes as well as MD simulations of the systems described above. The systems were structurally aligned to the substrate or group of residues with the smallest root-mean-square fluctuation (RMSF) values. Hydrogen bonds were inspected by protein donor—protein acceptor distance measuring.

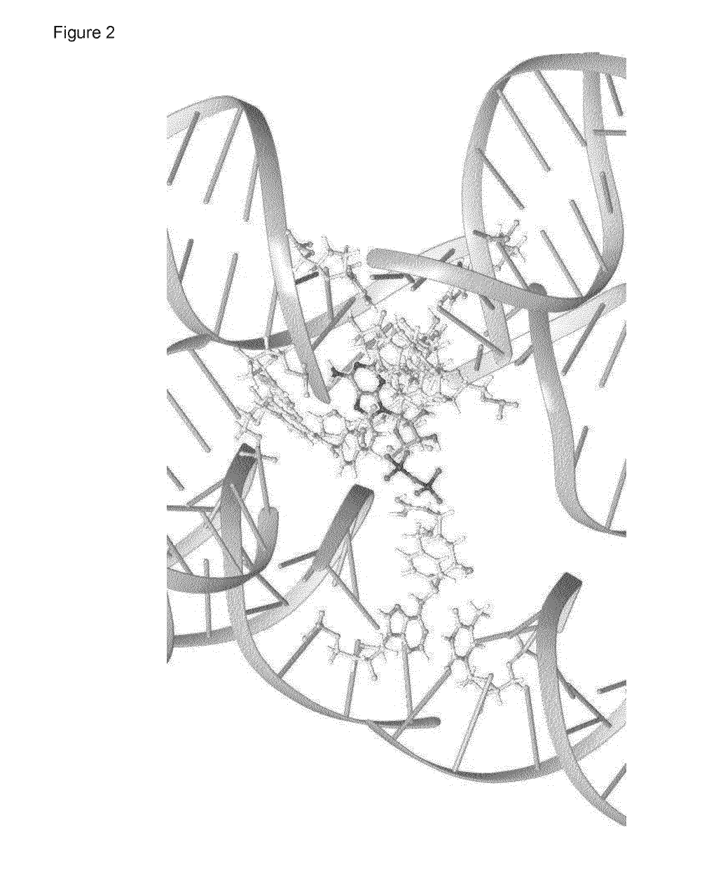

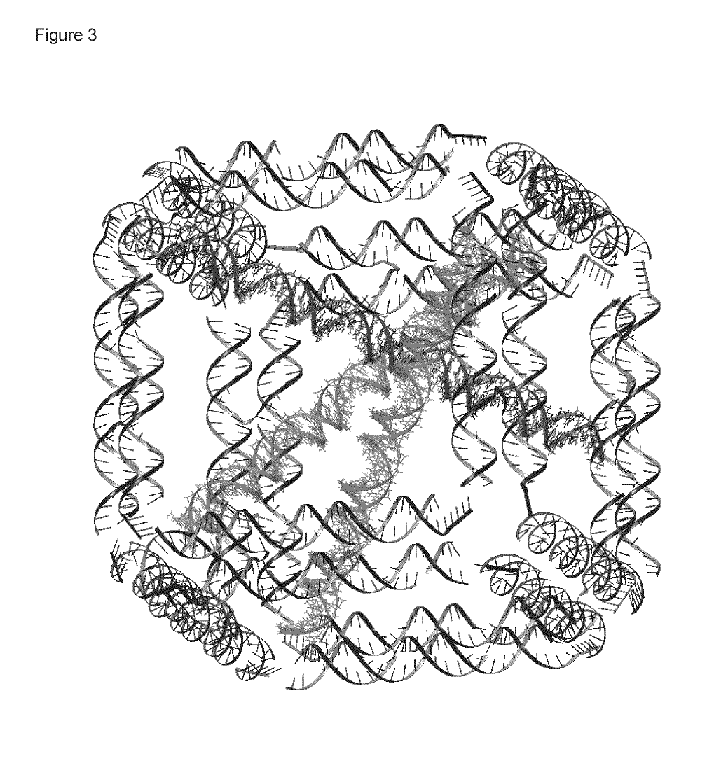

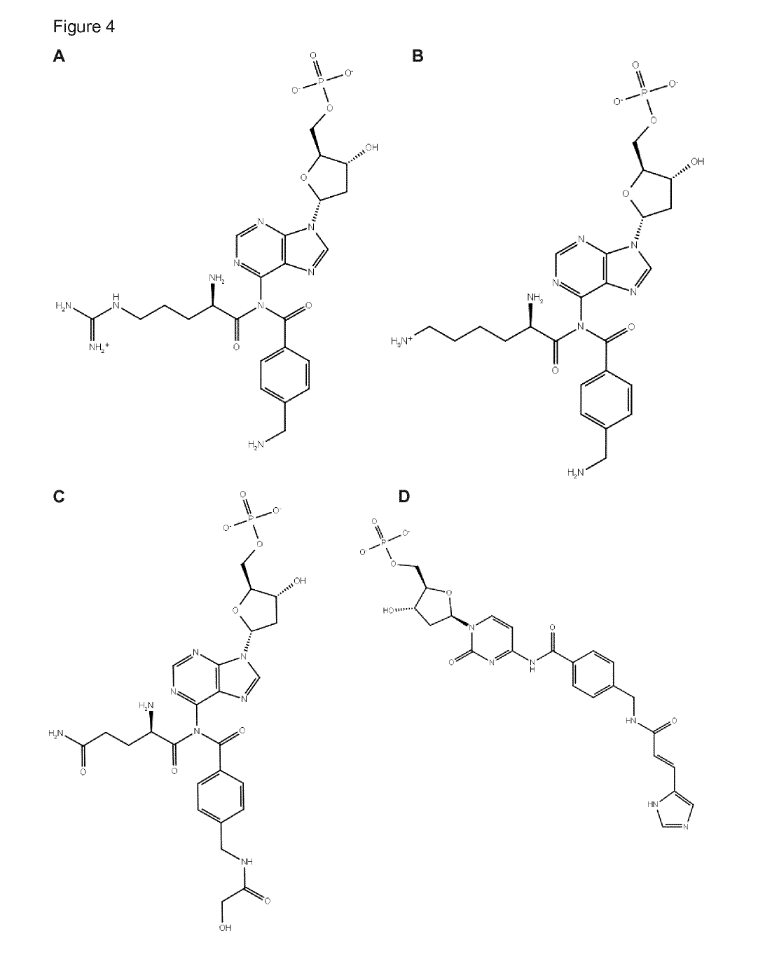

Obtained information on type and substrate-relative position of amino acids involved in ATP/ADP+PO4 binding were used in the creation of a binding site for ADP/ATP inside of DNA nanostructure using Maestro. The DNA nanostructure was created in three separate parts, FIGS. 2-4) DNA box, a cubical structure made from single and double stranded DNA maintaining the structural integrity of whole nanostructure. II) Inner scaffold, a double stranded DNA structure made of three separated DNA helices connected on each end with the DNA box. Intermolecular distance of inner scaffold helices has been made shortest in the area of substrate binding. III) Substrate binding site, group of modified nucleotides specifically chosen and positioned on strands in the inner scaffold with the purpose of emulating ATP binding site residues of the FlaI protein complex.

### Example 3

### Evaluation of Structure and Function of the Nanostructures

The correct formation of the nanostructure is evaluated using e.g. electron microscopy or cryo-electron tomography. The functionality is evaluated by addition of e.g. a substrate to the nanostructure and the incorporation of fluorescently labelled nucleotides into the nucleic acid strands. Thus, conformational changes of the structure in the presence of a substrate can be monitored under a fluorescence microscope if the hydrolysis of the substrate is successfully performed. In the case of archeal rotary motor, the correct activity can be confirmed if one fluorescently labelled staple strand incorporated into the outer ring rotates clockwise and another fluorescently labelled staple strand incorporated into the inner e.g. disc or ring does not rotate or rotates counter-clockwise. However, the correct function of a nanostructure using a fluorescence microscope can only be determined in motile structures. The activity of nanostructures performing other enzymatic reactions such as chemical modifications of a substrate has to be assessed using other methods which are known by the person skilled in the art such as e.g. spectrophotometry and activity-based protein profiling (Willems et al., Bioconjug Chem., 25(7):1181-91 (2014)).

1. A nanostructure comprising a nucleic acid scaffold and at least one functional core forming a catalytic center, wherein the functional core comprises a nucleic acid molecule with at least one chemically modified nucleotide.

2. The nanostructure of claim 1, wherein the scaffold is a two-dimensional or three-dimensional shape selected from the group consisting of a sheet, square, rectangle, nanotube, cylinder, ring, disc, ribbon, box, cube, pyramide, cross and rod.

3. The nanostructure of claim 1, wherein the scaffold is a DNA scaffold.

4. The nanostructure of claim 1, wherein the scaffold is an RNA scaffold.

5. The nanostructure of claim 3, wherein the DNA scaffold is assembled by a single-stranded DNA backbone chain and/or at least 50 single-stranded DNA staple chains.

6. The nanostructure of claim 4, wherein the RNA scaffold is assembled by a single-stranded RNA backbone chain and/or at least 50 single-stranded RNA staple chains.

7. The nanostructure of claim 5, wherein the backbone chain comprises at least 1,000 nucleotides.

8. The nanostructure of claim 5, wherein the staple chains comprise at least 30 nucleotides.

9. The nanostructure of claim 1, wherein the at least one functional core is bound to the nucleic acid scaffold via a staple chain.

10. The nanostructure of claim 1, wherein the at least one functional core comprises a catalytic center of an ATP-driven motor or a catalytic center of an enzyme.

11. The nanostructure of claim 1, wherein the at least one functional core is embedded in an interior space of a nucleic acid scaffold having the shape of a nanotube, cylinder, pyramid, or box.

12. The nanostructure of claim 1, wherein the at least one functional core comprises a catalytic center with at least 5 amino acid residues and/or amino acid analogs.

13. The nanostructure of claim 1, comprising at least three functional cores forming the catalytic center of an ATPase, preferably.

14. The nanostructure of claim 13, wherein the nucleic acid scaffold emulates one or more structural or functional proteins of a flagellum or archaellum or fragments thereof.

15. The nanostructure of claim 14, further comprising one or more structural or functional proteins of a flagellum or archaellum.

16. The nanostructure of claim 1, wherein the nucleic acid molecule is a chemically modified nucleotide with one or more amino acid or amino acid analog residue(s).

17. The nanostructure of claim 10, wherein the at least one functional core is selected from the group consisting of an archaeal rotary motor, an ion pump, a light-harvesting complex, or a photosystem.

18. The nanostructure of claim 13, wherein the functional core comprises the ATPase FlaI embedded in an interior space of a nucleic acid scaffold having the shape of a nanotube, a cylinder, a pyramid, or a box.

19. The nanostructure of claim 14, wherein the nucleic acid scaffold emulates one or more proteins selected from the group consisting of FlaI, FlaX, FlaH, FlaJ, and fragments of one of the foregoing proteins.

20. The nanostructure of claim 15, wherein the one or more proteins are selected from the group consisting of FlaI, FlaX, FlaH, FlaJ, and fragments of one of the foregoing proteins.


SEQUENCE LISTING (4 sequences)

SEQ ID NO: 1 - protein, 513 aa, Sulfolobus acidocaldarius
MSFVEDYLTKLQERPTIIENPNILKGSKIFNAIYRVDDFVYIHIQSIKSEDGYNQYNVIEPPRPTHDEMEEIEEKFALSIGDKEPPEDTKEKEKLIRSILDKILLRMRLSVPKEYVIYHFIRDKLYTGSLEPLIRDPYIEDISIPGLGHVYIVHKVFGPMRTSIKFENYEELDNLIVSLSEKSYRPVSHNRPVVDASLPDGSRVNFVYGVDISRRGSNLTVRKFSRVPTSITQLIMFGTLSSMMAAYIWTMLDEGMNLFVCGETASGKTTTLNAITAFIPPNLKIVTIEDTPELTVPHSNWVAEVTRETGGEGTIKLFDLLKAALRQRPNYILVGAIRDKEGNVAFQAMQTGHSVMATFHAANITTLIQRLTGYPIEVPKSYINNLNIALFQTALYDKKGNLIRRVVEVDEIIDIDPVTNDVVYIPAFTYDSVQDKMLFAGKGSSYLIENKIAVKRGIDRRNIGLLYDELQMRSRFLNLLVEKKIFNYYDVWDYILRARQMGLEEAIKYVSNI

SEQ ID NO: 2 - DNA, 47 bp, Artificial Sequence
  FunC1
acgcgatgcatcgactagccttaatatggcggatatgcgcatgccgg

SEQ ID NO: 3 - DNA, 32 bp, Artificial Sequence
  FunC2
agctttagcgcggcgaggctagcgcttgatcg

SEQ ID NO: 4 - DNA, 51 bp, Artificial Sequence
  FunC3
agatctatgcgcgatcggcgatagcgccgatcgatcgcgatcgatgcgact